(19) United States
(12) Patent Application Publication
(10) Pub. No.: US 2004/0029100 A1
(43) Pub. Date: Feb. 12, 2004

(54) Rapid screen to identify p-glycoprotein substrates and high affinity modulators

(76) Inventors: Frances J. Sharom, Guelph (CA); Liu Guelph, Ontario (CA)

Correspondence Address: BERESKIN AND PARR, SCOTIA PLAZA, 40 KING STREET WEST-SUITE 4000 BOX 401, TORONTO, ON, M5H 3Y2, CA

(21) Appl. No.: 10/363,260

(22) PCT Filed: Sep. 11, 2001
(86) PCT No.: PCT/CA01/01264

(30) Foreign Application Priority Data
Sep. 11, 2000 (US) 60231710

(51) Int. Cl. C12Q 1/00; G01N 33/53; G01N 33/567

(52) U.S. Cl. 435/4; 435/7.2

(57) ABSTRACT

A novel method for identifying compounds that interact with P-glycoprotein that involves measuring the quenching of intrinsic tryptophan fluorescence is described. The method has many uses including (1) it can be used to screen drugs for their ability to interact with P-glycoprotein; (2) it can be used to screen for high affinity modulators of P-glycoprotein; and (3) it can be used to screen drugs that are potential hazards when used in combination with the modulators and (4) it can be used in methods of conducting target discovery/screening businesses.

### FIELD OF THE INVENTION

The present invention relates to methods of assaying compounds that interact with proteins, in particular methods of identifying compounds that interact with P-glycoprotein.

### BACKGROUND OF THE INVENTION

The ABC superfamily is a large group of proteins responsible for movement of substrates across biological membranes, driven by the energy of ATP hydrolysis (1, 2). The substrates moved by ABC proteins are as diverse as ions (e.g. CFTR, a chloride ion channel), and large proteins (e.g. hemolysin B exports hemolysin, a 80 kDa toxic protein). Some family members are importers, such as the bacterial histidine and maltose permeases, while others are exporters, and CFTR is a channel, rather than a transporter. The P-glycoprotein multidrug transporter (Pgp) exports an astonishing variety of hydrophobic natural products, drugs and peptides from mammalian cells, powered by the energy of ATP hydrolysis at its two nucleotide binding (NB) domains. Its physiological role is thought to involve protection against toxic xenobiotics by efflux or secretion of these compounds at the lumenal surfaces of the gut, kidney tubules and bile ductules, and its presence in the endothelial cells of the brain appears to make a major contribution to the blood brain barrier (3). Pgp also plays an important role in the multidrug resistance (MDR) displayed by many human tumours, and is an important factor in predicting the outcome of chemotherapy treatment (4, 5).

The application of fluorescence techniques to the study of the structure and function of Pgp has proved very fruitful over the past few years (for reviews, see 6, 7). Biophysical approaches such as this have been made possible by the development of methods for the isolation of sub-milligram amounts of purified Pgp with high levels of ATPase and drug transport activity. Experiments on purified Pgp labelled with the extrinsic fluorophore, MIANS, at Cys residues within the NB domains have led to important insights into the molecular architecture of the protein. For example, quenching experiments showed the existence of cross-talk between the catalytic sites in the NB domains, and the drug binding sites, which are thought to be made up by the membrane-spanning regions of the protein within the lipid bilayer (8). Binding to MIANS-labeled Pgp of nucleotides, and drug or peptide substrates, takes place with apparently normal affinity, however, the transporter is catalytically inactive (8, 9). This potential limitation might be overcome by the use of intrinsic Trp fluorescence of the catalytically active protein as a reporter technique. Trp fluorescence studies have proved invaluable for studying the interaction of a variety of ATPases and ATP-utilizing enzymes with their substrates, including the FₒF₁-ATPase (10), plasma membrane Ca²⁺-ATPase (11), sarcoplasmic reticulum Ca²⁺-ATPase (12), the DnaB helicase (13), and phosphofructokinase (14), as well as membrane transporters, such as melibiose permease (15) and lactose permease (16).

Aromatic amino acid chains, such as those of Trp, Tyr and Phe, were found to be over-represented in the TM (transmembrane) regions of Pgp (17). It has been proposed that these residues, which are highly conserved across the Pgp family, may be involved in substrate recognition and interaction, and a model has been suggested whereby substrates with aromatic rings, such as rhodamine 123, can intercalate between the side chains of aromatic-rich TM helices at the protein-lipid interface (17). In this regard, Neyfakh and co-workers have recently proposed a working model for multidrug transporters and multidrug-binding proteins, in which polyspecific recognition of multiple substrates is based on complementarity of the molecular surfaces via Van der Waal's interactions and overall steric fit (18, 19). Since many Pgp substrates contain aromatic rings, the involvement of aromatic side chains in these interactions seems likely. In addition, peptides selected from a phage display library for their ability to bind MDR drugs with high affinity were found to contain the consensus motif WXXW (20). Aromatic side chains such as the indole ring of Trp may thus play an important structural role in substrate recognition by Pgp, and their fluorescence properties may be sensitive to the process of substrate binding.

To date, little work has been conducted on the intrinsic fluorescence of Pgp. Sonveaux et al. reported data for quenching of the Trp fluorescence of purified Pgp with iodide ion (21), and more recently, used changes in acrylamide quenching susceptibility as an indicator of conformational changes taking place on binding of various anthracycline derivatives and ATP (22). The fluorescence properties of the single Trp residue present within the C-terminal NB domain (NB2) of mouse Pgp were examined in a separately expressed domain, both as a fusion protein and after cleavage (23). Although purified NB2 was catalytically inactive, it bound fluorescent ATP and ADP derivatives, and its single Trp residue was highly quenched by binding of fluorescent nucleotides (23), steroids (24) and flavonoids (25).

### SUMMARY OF THE INVENTION

A comprehensive study of the intrinsic fluorescence properties of highly purified, catalytically active P-glycoprotein (Pgp) in its native state has been performed. Both steady-state and lifetime experiments were used to characterize the environment of the 11 Trp residues within the protein, and the changes that take place in their fluorescence properties on substrate binding. It has been shown that the Trp fluorescence of Pgp is highly sensitive to the binding of nucleotides, both unmodified and fluorescent derivatives, and also to binding of a wide variety of drugs, modulators and hydrophobic peptides that serve as substrates for the transporter. It seems likely that Trp residues are directly involved in binding of some drug substrates, and are probably in close proximity to the regions of the transporter involved in recognition of others. As a result, a novel method for identifying compounds that interact with P-glycoprotein involving the quenching of intrinsic tryptophan fluorescence has been developed. The method is advantageous over the existing methods in several respects. In particular, the method is simple, universal in that it can identify both substrates and modulators of P-glycoprotein and it also provides a quantitative assessment of the ability of a compound to interact with P-glycoprotein.

Accordingly, the present invention provides a method of assaying for compounds that interact with P-glycoprotein comprising measuring the intrinsic tryptophan fluorescence of P-glycoprotein in the presence of varying concentrations of the compound, and determining a constant describing the interaction of the compound with P-glycoprotein.

In a preferred embodiment of the present invention, the constant that is extracted from the fluorescence data is the dissociation constant. Accordingly, the present invention provides a method of determining the dissociation constant (K_{d}) of a compound for P-glycoprotein comprising measuring the intrinsic tryptophan fluorescence of P-glycoprotein in the presence of varying concentrations of the compound, and determining the dissociation constant.

In embodiments of the present invention, the constant, in particular the dissociation constant (K_{d}) is determined by fitting the fluorescence measurements to an equation describing binding to a single site or multiple sites and extraction of the constant.

The present invention further provides a method of assaying for compounds that interact with P-glycoprotein comprising:

(a) providing a sample of P-glycoprotein;

(b) adding a first concentration of a compound to the sample of P-glycoprotein and measuring the intrinsic tryptophan fluorescence;

(c) repeating steps (a) and (b) with a second concentration of the compound; and

(d) determining the dissociation constant (K_{d}) for the binding of the compound to P-glycoprotein, wherein the K_{d} indicates the interaction of the compound with P-glycoprotein.

In a preferred embodiment of the present invention, the intrinsic tryptophan fluorescence of P-glycoprotein is measured in the presence of varying concentrations of the compound and one or more lipids, preferably one or more phospholipids.

The method of the invention has many uses including (1) it can be used to screen drugs for their ability to interact with P-glycoprotein; (2) it can be used to screen for high affinity modulators of P-glycoprotein; (3) it can be used to screen drugs that are potential hazards when used in combination with the modulators and (4) it can be used in methods of conducting target discovery/screening businesses.

The method of the invention may be used in standard or high-throughput screening formats.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1 is a schematic diagram showing the location of the 11 Trp residues within the hamster class I Pgp, based on a topology model similar to that proposed for the murine mdr3 protein (34). The N-half of the transporter contains 8 Trp residues; Trp44 in the N-terminal tail, Trp133, Trp229 and Trp312 in TM2, TM4 and TM5 respectively, Trp159 in the first cytoplasmic loop, Trp209 in the second extracellular loop, and Trp695 and Trp705 in the linker region immediately following NB1. The C-half of Pgp contains 3 Trp residues; Trp800 in cytoplasmic loop 3, Trp852 in extracellular loop5 and Trp1105 between the Walker A and B motifs of NB2.

FIG. 2 shows corrected fluorescence emission spectra for purified native Pgp (100 μg/mL) in buffer containing 2 mM CHAPS and 0.5 mg/mL PMPC (—), and following treatment with 6M guanidine HCl (GuHCl; - - -), compared with 30 μg/mL of the soluble Trp analogue NATA (...). Fluorescence emission was recorded at 22° C. following excitation at 290 nm.

FIG. 3(A) shows the fluorescence decay of purified Pgp in buffer containing 2 mM CHAPS and 0.5 mg/mL PMPC. Fluorescence emission was collected at 350 nm following excitation at 295 nm. Double exponential analysis recovered the following values: a₁=0.32, τ₁=0.68 ns, a₂=0.28, τ₂=4.2 ns, a₁/a₂=1.1, χ²=1.08. FIG. 3(B) shows the lifetime distribution analysis with ESM of the Pgp decay shown in (A). Lifetime values averaged over respective peaks are 0.73 ns and 4.2 ns, the ratio of integrated amplitudes a₁/a₂=1.0, χ²=1.07.

FIG. 4 shows the Stern-Volmer plots for quenching of the intrinsic Trp fluorescence of Pgp by acrylamide (A, B) and I⁻ (C, D). Expanded plots for Pgp quenching are shown in (B) for acrylamide and (D) for I⁻. Aliquots of the appropriate quencher were added to a 100 μg/mL solution of purified Pgp in buffer containing 2 mM CHAPS and 0.5 mg/mL PMPC. Parallel experiments were carried out with 30 μg/mL of NATA. Fluorescence emission at 330 nm was recorded at 22° C. following excitation at 290 nm. Where not visible, error bars are contained within the symbols.

FIG. 5 shows the acrylamide quenching of the intrinsic Trp fluorescence of Pgp in the presence of various nucleotides and drug substrates. Aliquots (5 μL) of a 5 M solution of acrylamide were added at 22° C. to 0.5 mL of a 100 μg/mL solution of purified Pgp in buffer containing 2 mM CHAPS and 0.5 mg/mL PMPC. (A) Pgp alone (●), and in the presence of 2 mM ATP (●) or 2 mM AMP-PNP (Δ). (B) Pgp alone (●), or in the presence of 10 μM vinblastine (VBL, ●) or 10 μM vinblastine+2 mM AMP-PNP (Δ). (C) Pgp alone (●), or in the presence of 10 μM cyclosporin A (CsA, ●) or 10 μM cyclosporin A+2 mM AMP-PNP. (D) Pgp alone (●, or in the presence of 20 μM daunorubicin (DAU, ●) or 10 μM daunorubicin+2 mM AMP-PNP. Fluorescence emission at 330 nm was recorded at 22° C. following excitation at 290 nm. Where not visible, error bars are contained within the symbols. Values for the Stern-Volmer constants, Kₛᵥ, were determined from the slopes of the plots.

FIG. 6 shows the effect of nucleotide binding on the intrinsic Trp fluorescence of Pgp. Increasing concentrations of (A) ATP, (B) AMP-PNP, (C) TNP-ATP and (D) TNP-ADP were added at 22° C. to a 100 μg/mL solution of purified Pgp in buffer containing 2 mM CHAPS and 0.5 mg/mL PMPC. Fluorescence emission at 330 nm was recorded at 22° C. following excitation at 290 nm. Where not visible, error bars are contained within the symbols.

FIG. 7 shows the effect of binding of various drugs and hydrophobic peptides on the intrinsic Trp fluorescence of Pgp. Increasing concentrations of (A) vinblastine, (B) cyclosporin A, (C) LY294002 and (D) trifluoperazine were added at 22° C. to a 100 μg/mL solution of purified Pgp in buffer containing 2 mM CHAPS and 0.5 mg/mL PMPC. Fluorescence emission at 330 nm was recorded at 22° C. following excitation at 290 nm. The quenching curve for cyclosporin A was essentially monophasic, and was fitted to an equation describing binding to a single type of site (indicated by the solid lines). Vinblastine quenching showed some biphasic character, and could be fitted to equations for either one or two sites; one-site fitting is shown here. For LY294002 and trifluoperazine, clearly biphasic quench curves were obtained, which were fitted to equations for two binding sites, one of high and one of low affinity (see ref. (32)). Where not visible, error bars are contained within the symbols.

FIG. 8 shows the quenching of the intrinsic Trp fluorescence of Pgp by sequential addition of nucleotides and drugs. In (A) and (B), increasing concentrations of vinblastine (VBL) and TNP-ATP were added sequentially, either vinblastine first (A), or TNP-ATP first (B), to a 100 μg/mL solution of purified Pgp in buffer containing 2 mM CHAPS and 0.5 mg/mL PMPC. Fluorescence emission at 330 nm was recorded at 22° C. following excitation at 290 nm. (C) and (D) show a similar series of experiments using sequential titration of daunomycin (DAU) and TNP-ATP. Where not visible, error bars are contained within the symbols.

FIG. 9 shows the relationship between the value of the maximal Trp quenching parameter, ΔFₘₐₓ, and the spectral overlap integral, J, for 12 different Pgp substrates; trifluoperazine (TFL); LY294002 (LY), daunorubicin (DAU); doxorubicin (DOX); quinine (QUN); quinidine (QDN); vinblastine (VBL); rhodamine 6G (Rho6G); tetramethylrosamine (TMR); rhodamine 123 (Rho123); TNP-ATP; and colchicine (CLC).

### DETAILED DESCRIPTION OF THE INVENTION

### I. Methods of the Invention

As hereinbefore mentioned, the present inventors have developed a novel method for assaying compounds that interact with P-glycoprotein.

Accordingly, the present invention provides a method of assaying for compounds that interact with P-glycoprotein comprising measuring the intrinsic tryptophan fluorescence of P-glycoprotein in the presence of varying concentrations of the compound, and determining a constant describing the interaction of the compound with P-glycoprotein.

In a preferred embodiment of the present invention, the constant that is extracted from the fluorescence data is the dissociation constant. Accordingly, the present invention provides a method of determining the dissociation constant (K_{d}) of a compound for P-glycoprotein comprising measuring the intrinsic tryptophan fluorescence of P-glycoprotein in the presence of varying concentrations of the compound. and determining dissociation constant.

In embodiments of the present invention, the constant, in particular the dissociation constant (K_{d}) is determined by fitting the fluorescence measurements to an equation describing binding to a single site or multiple sites and extraction of the constant.

In a preferred embodiment, the dissociation constant, K_{d}, is measured by calculating the percentage (%) quenching of the fluorescence at each concentration of the compound followed by fitting the data to an equation describing binding to a single site or multiple sites and extraction of the dissociation constant.

The present invention further provides a method of assaying for compounds that interact with P-glycoprotein comprising:

(a) providing a sample of P-glycoprotein;

(b) adding a first concentration of a compound to the sample of P-glycoprotein and measuring the intrinsic tryptophan fluorescence;

(c) repeating steps (a) and (b) with a second concentration of the compound; and

(d) determining the dissociation constant (K_{d}) for the binding of the compound to P-glycoprotein, wherein the K_{d} indicates the interaction of the compound with P-glycoprotein.

Compounds with a K_{d} of greater than about 10 μM, preferably greater than about 100 μM, are considered to have a low interaction (or affinity) with P-glycoprotein while compounds with a K_{d} of less than about 10 μm, preferably about 1 μM, are considered to have a high interaction (or affinity) with P-glycoprotein.

The sample of P-glycoprotein used in the methods of the invention is initially preferably free of ATP or lipids and is a purified preparation of P-glycoprotein. Any source of P-glycoprotein from any species may be used in the assay of the invention, including human P-glycoprotein, from either multidrug-resistant cell lines, or from heterologous expression systems, such as (but not limited to) yeast and bacteria. Preferably the source of P-glycoprotein is a mammal, including, but not limited to human, monkey, mouse, rabbit, rat or hamster. In one embodiment, the P-glycoprotein is purified from the multi-drug resistant Chinese hamster ovary cell line CH^{R}B30.

In a preferred embodiment of the present invention, the intrinsic tryptophan fluorescence of P-glycoprotein is measured in the presence of varying concentrations of the compounds and one or more lipids. Lipids that can be used in the method of the invention are preferably phospholipids, including, but not limited to, egg phosphatidylcholine (egg PC), dimyristoylphosphatidylcholine (DMPC), dipalmitoylphosphatidylcholine (DPPC), and palmitoylmyristoylphosphatidylcholine (PMPC). Preferably the lipid is PMPC.

The P-glycoprotein is preferably titrated with at least 5 concentrations of the test compound, more preferably, at least 10 concentrations of the test compound, even more preferably at least 15 concentrations of the test compound.

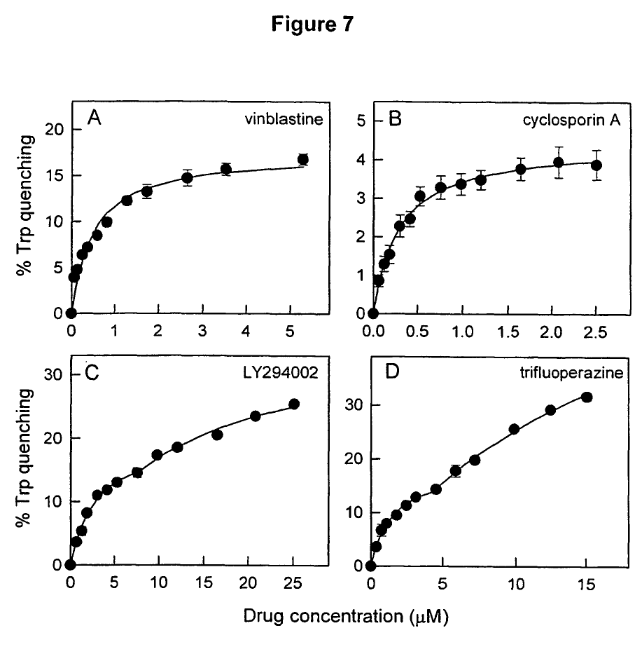

Using the method of the present invention, the inventors have determined the K_{d} value for over 30 compounds. The results are presented in Table 2 which demonstrates that the Kd range can vary from 62 nM (very high affinity) to 75 μM (very low affinity). The typical tryptophan quenching curves for four of these compounds are shown in FIG. 7.

### II. Uses of the Method

The novel method of the invention has utility in many applications some of which are summarized below. It is to be understood that the invention includes all possible uses of the method of the invention.

(a) Screening Drugs in Development

In one aspect, the method of the invention can be used to screen drugs in development for their ability to interact with P-glycoprotein. In particular, it has been shown that the higher affinity (i.e. lower Kd) a drug has for P-glycoprotein the less likely it is to be absorbed in the intestine and therefore the less efficient the drug will be. It appears that the poor oral bioavailability of many drugs is due to their lack of uptake into intestinal cells because of efflux into the gut lumen by P-glycoprotein. Similarly, the poor penetration of many drugs into the brain can also be attributed to the presence of P-glycoprotein.

Therefore, by using the method of the present invention, companies involved in the development of drugs can test the ability of the drug to interact with P-glycoprotein which will provide them with an indication of whether or not the drug will be of clinical significance early in the developmental process i.e., before large sums of money have been expended.

Accordingly, the present invention provides a method of determining if a compound is a good drug candidate comprising measuring the intrinsic tryptophan fluorescence of P-glycoprotein in the presence of varying concentrations of the compound, fitting the fluorescence measurements to an equation describing binding to a single site or multiple sites and extraction of a constant describing the interaction of the compounds with P-glycoprotein, wherein the value of the constant indicates whether the compound is a good drug candidate.

Preferably the constant that is extracted from the fluorescence data is the dissociation constant K_{d}. Accordingly, the present invention provides a method of determining if a compound is a good drug candidate comprising:

(a) providing a sample of P-glycoprotein in the presence;

(b) adding a first concentration of the compound to the sample of P-glycoprotein and measuring the intrinsic tryptophan fluorescence;

(c) repeating steps (a) and (b) with a second concentration of the compound; and

(d) determining the dissociation constant (K_{d}) for the binding of the compound to P-glycoprotein, wherein the value of K_{d} indicates whether the compound is a good drug candidate.

In a preferred embodiment of the present invention, the intrinsic tryptophan fluorescence of P-glycoprotein is measured in the presence of varying concentrations of the compound and one or more lipids, preferably, one or more phospholipids.

Compounds with a K_{d} of greater than about 10 μM, preferably greater than about 100 μM, are considered to be good drug candidates (i.e. are expected to have good bioavailability) while compounds with a K_{d} of less than about 10 μM, preferably about 1 μM, are considered to poor drug candidates (i.e. are expected to have poor bioavailability).

(b) Screening for High Potency Modulators

In another aspect, the method of the invention can also be used to screen for potential modulators which interact with P-glycoprotein with high affinities. Modulators (also known as chemosensitizers or reversal agents) block the pumping mechanism of P-glycoprotein by an as yet undefined mechanism. Some may act as alternate substrates (i.e. they are transported by the protein), others appear to bind P-glycoprotein, but are not transported. Several modulators are currently being tested in clinical trials on cancer patients. There is a need for development of very high affinity P-glycoprotein modulators that will be more efficacious.

The present invention therefore provides a method of identifying compounds that are high affinity P-glycoprotein modulators comprising measuring the intrinsic tryptophan fluorescence of P-glycoprotein in the presence of varying concentrations of the compound, fitting the fluorescence measurements to an equation describing binding to a single site or multiple sites and extraction of a constant describing the interaction of the compounds with P-glycoprotein, wherein the value of the constant indicates whether the compound is a potentially effective P-glycoprotein modulator.

Preferably the constant that is extracted from the fluorescence data is the dissociation constant K_{d}. Accordingly, the present invention provides a method of identifying compounds that are high affinity P-glycoprotein modulators comprising:

(a) providing a sample of P-glycoprotein;

(b) adding a first concentration of the compound to the sample of P-glycoprotein and measuring the intrinsic tryptophan fluorescence;

(c) repeating steps (a) and (b) with a second concentration of the compound; and

(d) determining the dissociation constant (K_{d}) for the binding of the compound to P-glycoprotein, wherein the value of K_{d} indicates if the compound is a potentially effective P-glycoprotein modulator.

In a preferred embodiment of the present invention, the intrinsic tryptophan fluorescence of P-glycoprotein is measured in the presence of varying concentrations of the compound and one or more lipids, preferably, one or more phospholipids

In further preferred embodiments, a K_{d} of less than about 10 μM, preferably less than about 1.0 μM, more preferably less than about 0.1 μM, indicates that the compound is a potentially effective P-glycoprotein modulator.

The invention extends to all compounds identified using the method of the invention and to compositions comprising a compound identified using a method of the invention and a pharmaceutically acceptable carrier. Further the invention includes methods of preparing a composition comprising determining whether a compound is a modulator of P-glycoprotein using a method according to the invention and admixing said compound with a pharmaceutically acceptable carrier.

(c) Screening for Drugs that are Potential Hazards when Used in Combination with Modulators

In another aspect, the method of the invention can be used to determine if a drug can be potentially hazardous when used in combination with a modulator. If high potency modulators come into more general use in treating cancer patients, a problem of potentially enormous importance will arise. Highly effective blocking of P-glycoprotein by high potency modulators (which has not yet been achieved with existing drugs) will permit highly effective uptake and brain penetration of any co-administered drugs that are P-glycoprotein substrates. This may result in high levels of toxicity. This proposal has been borne out by studies on P-glycoprotein knockout mice (which would simulate the end-point of total inhibition of P-glycoprotein function). Many otherwise safe, non-toxic (some over-the-counter) drugs show high levels of brain penetration, and in one case (the anti-helminthic drug, ivermectin), led to high mortality among the transgenic animals.

The method of the present invention can therefore be used to determine the potential danger of using a drug in combination with a P-glycoprotein modulator. Drugs that normally have affinity for P-glycoprotein will no longer be transported by P-glycoprotein in the presence of a modulator and will therefore be absorbed at higher levels than in the absence of the modulator. As a result, the method of the invention can be used to determine whether the drug interacts with P-glycoprotein with high affinity, in which case a potentially dangerous or toxic combination exists if it is used with a modulator.

Accordingly, the present invention provides a method of determining if a compound may be dangerous if used in combination with a modulator of P-glycoprotein comprising measuring the intrinsic tryptophan fluorescence of P-glycoprotein in the presence of varying concentrations of the compound, fitting the fluorescence measurements to an equation describing binding to a single site or multiple sites and extraction of a constant describing the interaction of the compounds with P-glycoprotein, wherein the value of the constant indicates whether the compound may be dangerous if used in combination with a modulator of P-glycoprotein.

Preferably the constant that is extracted from the fluorescence data is the dissociation constant K_{d}. Accordingly, the present invention provides a method of determining if a compound may be dangerous if used in combination with a modulator of P-glycoprotein comprising:

(a) providing a sample of P-glycoprotein;

(b) adding a first concentration of the compound to the sample of P-glycoprotein and measuring the intrinsic tryptophan fluorescence;

(c) repeating steps (a) and (b) with a second concentration of the compound; and

(e) determining the dissociation constant (K_{d}) for the binding of the compound to P-glycoprotein, wherein the value of K_{d} indicates if the compound may be dangerous if used in combination with a modulator of P-glycoprotein.

In a preferred embodiment of the present invention, the intrinsic tryptophan fluorescence of P-glycoprotein is measured in the presence of varying concentrations of the compound and one or more lipids, preferably, one or more phospholipids

In further preferred embodiments, a K_{d} of less than about 10.0 μM, preferably less than about 1.0 μM, more preferably less than about 0.1 μM, indicates that the compound is potentially dangerous if used in combination with a modulator of P-glycoprotein.

(d) High Through-Put Screening Assays

The method of the invention may also be adapted to allow high through-put screening of large numbers of compounds. High throughput measurements of K_{d} for many drugs may be made by recording the fluorescence readout using a fluorescence plate-reader (96 well or more), or a fluorescence imaging device. This has the added advantage of greatly reducing the sample size required to make the measurement, from the point of view of both P-glycoprotein and drug.

The methods of the invention may be used to screen a wide variety of compounds or compound libraries for interaction with P-glycoprotein. A library of potential compounds can be a synthetic combinatorial library (e.g., a combinatorial chemical library), a cellular extract, a bodily fluid (e.g., urine, blood, tears, sweat, or saliva), or other mixture of synthetic or natural products (e.g., a library of small molecules or a fermentation mixture).

A library of potential inhibitors can include, for example, amino acids, oligopeptides, polypeptides, proteins, or fragments of peptides or proteins; nucleic acids (e.g., antisense; DNA; RNA; or peptide nucleic acids, PNA); aptamers; or carbohydrates or polysaccharides. Each member of the library can be singular or can be a part of a mixture (e.g., a compressed library). The library can contain purified compounds or can be “dirty” (i.e., containing a significant quantity of impurities).

Commercially available libraries (e.g., from Affymetrix, ArQule, Neose Technologies, Sarco, Ciddco, Oxford Asymmetry, Maybridge, Aldrich, Panlabs, Pharmacopoeia, Sigma, or Tripose) can also be used with the methods of the invention.

(e) Target Discovery Business

Yet another aspect of the present invention provides a method of conducting a target discovery business comprising:

(a) providing one or more assay systems for identifying compounds by their ability to interact with P-glycoprotein, said assay systems using a method of the invention;

(b) (optionally) conducting therapeutic profiling of agents identified in step (a) for efficacy and toxicity in animals; and

(c) licensing, to a third party, the rights for further drug development and/or sales of compounds identified in step (a), or analogs thereof.

By assay systems, it is meant, the equipment, reagents and methods involved in conducting a screen of compounds for the ability to interact with P-glycoprotein using the method of the invention.

(f) Target Screening Business

A further aspect of the present invention provides a method of conducting a compound screening business comprising:

(a) providing one or more assay systems for screening compounds for their ability to interact with P-glycoprotein, said assay systems using a method of the invention; and

(b) providing, to a third party, the information identified in step (a) in exchange for compensation.

By assay systems, it is meant, the equipment, reagents and methods involved in conducting a screen of compounds for the ability to interact with P-glycoprotein using the method of the invention.

### III. Kits

The reagents suitable for carrying out the methods of the invention may be packaged into convenient kits providing the necessary materials, packaged into suitable containers. For example the reagents may include reagents for performing the fluorescence assay, such as an aliquot of P-glycoprotein, an aliquot of lipid, for example an aliquot of PMPC, and buffer, for example CHAPS buffer, and other reagents for performing the fluorescence assay.

With particular regard to assay systems packaged in “kit” form, it is preferred that assay components be packaged in separate containers, with each container including a sufficient quantity of reagent for at least one assay to be conducted. A preferred kit is typically provided as an enclosure (package) comprising one or more containers for the within-described reagents.

The reagents as described herein may be provided in solution, as a liquid dispersion or as a substantially dry powder, e.g., in lyophilized form. Usually, the reagents are packaged under an inert atmosphere.

Printed instructions providing guidance in the use of the packaged reagent(s) may also be included, in various preferred embodiments. The term “instructions” or “instructions for use” typically includes a tangible expression describing the reagent concentration or at least one assay method parameter, such as the relative amounts of reagent and sample to be admixed, maintenance time periods for reagent/sample admixtures, temperature, buffer conditions, and the like.

The following non-limiting examples are illustrative of the present invention:

### EXAMPLES

### Example 1

Materials and Methods

Materials. Acrylamide was obtained from Bio-Rad Laboratories (Mississauga, ON). KI was purchased from Fisher Scientific (Unionville, ON Canada). TNP-ATP, TNP-ADP, tetramethylrosamine and rhodamine 6G were supplied by Molecular Probes (Eugene, OR). 3-[(3-Cholamidopropyl)-dimethylammonio]-1-propane-sulfonate (CHAPS), disodium-ATP, disodium-ADP, 5′-adenylylimido-diphosphate (AMP-PNP), N-acetyl-L-tryptophanamide (NATA), guanidine hydrochloride, colchicine, vinblastine, daunorubicin, doxorubicin, trifluoperazine, quinine, quinidine, pepstatinA, valinomycin, LY294002, rhodamine123, and rhodamine B were purchased from Sigma Chemical Co. (St. Louis, Mo.). Cyclosporin A was provided by Pfizer Central Research (Groton, Conn.). Dr. Balázs Sarkadi (National Institute of Haematology and Immunology, Budapest, Hungary) supplied reversins 121 and 205 (26).

Purification of Pgp. Pgp was purified from plasma membrane of MDR CH^{R}B30 cells by a modification of the method previously described for CH^{R}C5 (27). Following the initial extraction of plasma membrane with CHAPS buffer (25 mM CHAPS/50 mM Tris-HCl/0.15M NaCl/5 mM MgCl₂/0.02% w/v NaN₃, pH 7.5), the detergent-insoluble pellet was solubilized in 15 mM CHAPS buffer, in a volume of 330 μL for each mg of plasma membrane protein originally used. Incubation and centrifugation resulted in a soluble supernatant (S₂) which was highly enriched in Pgp. Contaminating glycoproteins were removed from the S₂ fraction by passing through a concanavalinA-Sepharose 4B column (Pharmacia) equilibrated with 2 mM CHAPS buffer. The final product consisted of a solution of 9095% pure Pgp, at a concentration of 0.10.2 mg/mL, in 2 mM CHAPS buffer. The Pgp preparation was kept on ice and used within 24 h.

Steady-state Fluorescence Measurements. Steady-state fluorescence measurements were performed using a Spex Model DM3000 spectrofluorimeter (Spex Industries Inc., Edison, N.J.) at 22° C., with excitation and emission bandpass set to 4 nm. Most fluorescence studies were carried out on solutions of 100 μg/mL Pgp in 2 mM CHAPS buffer, in the presence of 0.5 mg/mL of the phospholipid PMPC (Avanti Polar Lipids, Alabaster, Ala.). PMPC was added as extruded 100 nm unilamellar vesicles (8). With some drugs, the use of a lipid was not required for the fluorescence measurements. The excitation and emission spectra were the average of three scans, and were corrected by subtracting control scans with the same phospholipid buffer in the absence of protein.

Fluorescence Lifetime Measurements. Fluorescence decays were measured with a PTI Model C-720 fluorescence lifetime instrument ((28), Photon Technology International Inc., Lawrenceville, N.J.) utilizing a proprietary stroboscopic detection technique (29). The system employed a PTI GL-330 pulsed nitrogen laser pumping a PTI GL-302 high-resolution dye laser. The dye laser output at 590 nm was frequency doubled to 295 nm with a GL-103 frequency doubler coupled to an MP-1 sample, compartment vis fiber optics. The emission was observed at 90° relative to the excitation via an M-101 emission monochromator and a stroboscopic detector equipped with a Hamamatsu 1527 photomultiplier. Fluorescence decays were analyzed with a PTI TimeMaster Pro analysis package using a discrete 1- to 4- exponential fit, 1- to 4-exponential global analysis, or lifetime distribution analysis by the Exponential Series Method (ESM) (30). The quality of fits was judged by chi-square values and weighed residuals.

Quenching Studies using Acrylamide and Iodide. Stock solutions of 5 M acrylamide, or 5M KI, were added as 5 μL aliquots in buffer to 0.5 mL of 100 μg/mL Pgp in 2 mM CHAPS buffer with 0.5 mg/mL PMPC. All quencher solutions were freshly prepared, and 0.1 mM Na₂S₂O₃ was added to the KI stock solution to prevent I₃ formation. Fluorescence emission was measured at 330 nm following excitation at 290 nm. Fluorescence intensities were corrected for dilution and scattering, and in control assays, KCl was added at the same concentration to correct for any ionic strength effects. Parallel experiments were carried out using NATA to assess quenching of Trp fluorescence by the same agents when Trp is completely accessible in aqueous solution. Quenching data were analyzed using the Stern-Volmer equation (31),

F₀/F=1+K_{SV}[Q]

where F₀ and F are the fluorescence intensities in the absence and presence of quencher, respectively, [Q] is the concentration of quenching agent, and K_{SV} is the Stern-Volmer quenching constant. In the case of a purely collisional quenching mechanism, a Stern-Volmer plot of F₀/F vs. [Q] gives a linear plot with a slope of K_{SV}. K_{SV}=k_{q}τ₀, where k_{q} is a collisional quenching rate constant and τ₀ is the fluorescence lifetime in the absence of quencher.

Quenching Studies using TNP-Nucleotide Derivatives and Drugs. Fluorescence quenching studies with TNP-nucleotide derivatives and various drugs and chemosensitizers were carried out on solutions of 100 μg/mL Pgp in 2 mM CHAPS buffer, typically with 0.5 mg/mL PMPC. Working solutions of TNP-derivatives, and non-peptide drugs and modulators were also prepared in 2 mM CHAPS with 0.5 mg/mL PMPC vesicles, whereas peptide-based drugs and modulators were dissolved in dimethylsulfoxide (DMSO). Final concentrations of DMSO in the fluorescence cuvette were <10%; DMSO alone showed no effects on Trp fluorescence of Pgp. Quenching experiments were performed by successively adding 5 μL aliquots of TNP-derivatives or drug solution to 500 μL of Pgp solution in a 0.5 cm quartz cuvette. After each addition, the sample was excited at 290 nm and the steady-state fluorescence emission was measured at 330 nm. Fluorescence intensities were corrected for dilution, scattering, and the inner filter effect as described elsewhere (8). Control titrations were performed with 30 μM NATA to assess the non-specific quenching of Trp fluorescence by TNP-nucleotide derivatives and drugs.

The experimental quenching data were computer-fitted to the following equation as described previously (8)(9):

$$( \frac{Δ F}{{F}_{0}} \times 100 ) = \frac{( \frac{Δ {F}_{max}}{{F}_{0}} \times 100 ) \times [ S ]}{{K}_{d} + [ S ]}$$

where (ΔF/F₀ 100) is the percent quenching (percent change in fluorescence relative to the initial value), following addition of substrate at a concentration [S], and K_{d} is the dissociation constant. Fitting was carried out using nonlinear regression with the Marquardt-Levenberg algorithm (SigmaPlot, SPSS Inc., Chicago, Ill.), and values of K_{d} and (ΔFₘₐₓ/F₀ 100) were extracted. For biphasic titration curves, each section of the binding curve was fitted to the above equation separately, as described previously (32).

Determination of the Spectral Overlap Integral. The spectral overlap integral (J) was determined using the integral equation:

$$J = \frac{\int {F}_{D} ( λ ) \cdot {}_{A} ( λ ) {λ}^{4} δλ}{\int {F}_{D} ( λ ) δλ}$$

where F_{D} is the fluorescence intensity per unit wavelength interval in the presence of the potential donor only, ε_{A} is the molar extinction coefficient of the potential acceptor, and λ is the wavelength in cm. The Trp fluorescence emission spectra of Pgp was recorded using excitation at 290 nm, and the absorption spectra of TNP-nucleotide derivatives and drugs were recorded using a computer-interfaced Perkin-Elmer Lambda 6 UV/visible spectrophotometer (Perkin-Elmer, Norwalk, Conn.) with both sample and reference cells at 22° C. J was calculated from the spectral data using a computer program designed solely for that purpose by Dr. Uwe Oehler (Department of Chemistry and Biochemistry, University of Guelph).

The quantum yield of Pgp Trp fluorescence, Q, was determined relative to NATA as a standard. Both the sample and standard had the same absorbance value of 0.095 at 290 nm. The quantum yield of Pgp was calculated using the equation:

$${Q}_{Pgp} = \frac{{F}_{Pgp}}{{F}_{NATA}} \times {Q}_{NATA}$$

where Q_{NATA}, the quantum yield of NATA, is known to be 0.14, and F_{Pgp} and F_{NATA} are the integrals of the fluorescence emission of Pgp and NATA in the wavelength range 310 to 500 nm.

Results

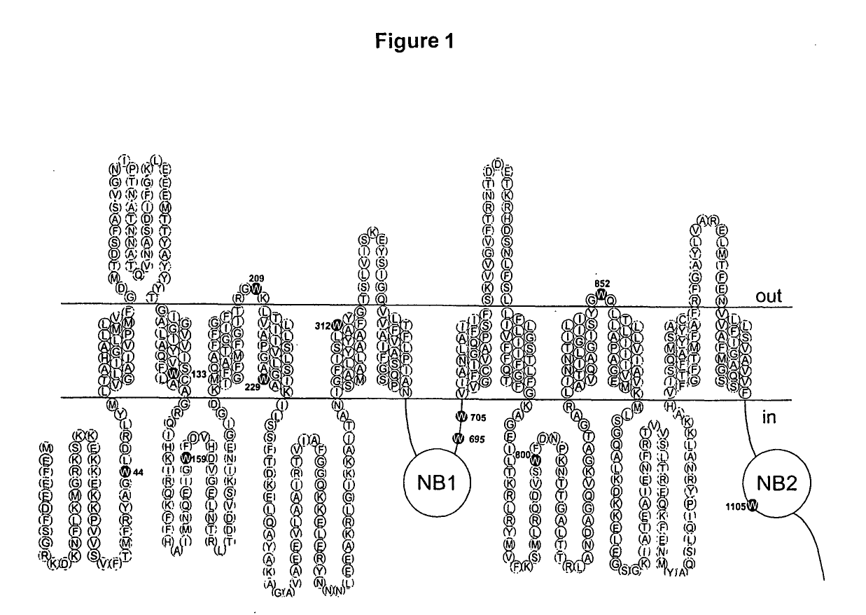

Location of Trp residues within Hamster Pgp. Hamster Pgp (the Pgp1 gene product (33)) contains 11 Trp residues distributed throughout the protein. The schematic diagram shown in FIG. 1 displays the placement of these Trp residues within hamster Pgp1, based on the proposed topology for the mouse mdr3 homologue (34). The N-terminal half of Pgp contains 7 Trps, and the C-terminal half contains 4 residues. Three residues in the N-half (Trp133, Trp229 and Trp312) are located within putative TM segments 2, 4 and 5, respectively. Trp44 is positioned in the N-terminal tail, and Trp159 and Trp209 in an intracellular and extracellular loop, respectively. Two residues (Trp695 and Trp705) are found in the linker region following NB1, just upstream of TM7. In the C-half, residues 800 and 852 are located in an intracellular and extracellular loop, respectively, and Trp1105 is situated within NB2, between the Walker A and B motifs. The position of these Trp residues is highly conserved in both the human (MDR1) and mouse (mdr3) homologues.

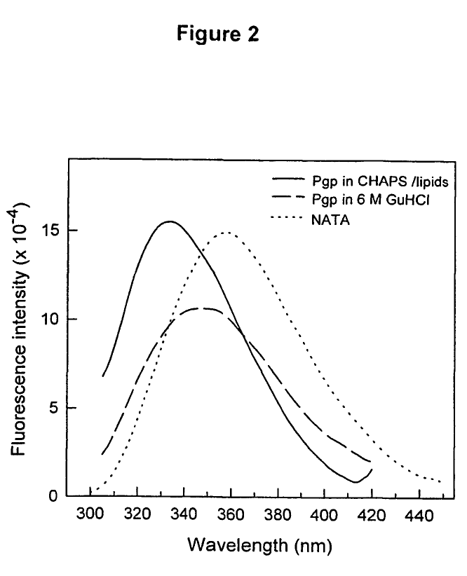

Characterization of Intrinsic Trp Fluorescence of Purified Pgp. Highly purified, catalytically active Pgp was isolated from MDR CH^{R}B30 Chinese hamster ovary cells. Steady-state and lifetime fluorescence studies were carried out in order to characterize the properties and environment of the Trp residues. Most measurements were carried out in the presence of the phospholipid PMPC, which gave the lowest background fluorescence of several lipids tested under the experimental conditions. Pgp has previously been successfully reconstituted into proteoliposomes of PMPC in fully active form (35). Some drugs did not require the presence of phospholipids to observe reproducible high-level quenching of Trp fluorescence induced by substrate binding. The emission spectrum of purified Pgp (λₑₓ=290 nm) exhibited a maximum at 333 nm, indicating that the Trp residues of Pgp are located in a highly nonpolar environment compared to the soluble Trp analogue, NATA, which displayed an emission maximum at 356 nm (FIG. 2). Pgp underwent a large red shift (14 nm) in fluorescence emission to 347 nm following denaturation of the protein in 6 M guanidine HCl (FIG. 2), which would largely unfold the protein. Several of the Trp residues in Pgp are located in extramembranous regions of the protein and are thus potentially highly accessible to their aqueous surroundings. The relatively hydrophobic environment indicated by the emission maximum suggests that these residues either do not contribute to the observed fluorescence emission (i.e. they are internally quenched), or they are present in nonpolar regions within tightly folded protein domains.

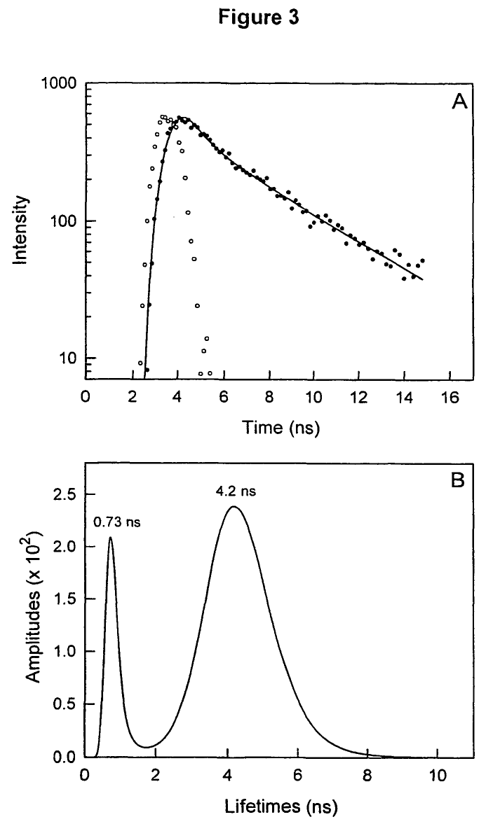

Fluorescence Lifetime of Trp Residues in Pgp. The fluorescence decay of NATA could be adequately described by a single exponential function with a lifetime, τ, of 2.6 ns (Table 1). However, as shown in FIG. 3A, two exponentials were required to obtain a satisfactory fit for the Pgp Trp decay. The longer lifetime component of ˜4 ns made the major contribution to the overall fluorescence compared to the shorter component, which had a lifetime of 0.6-1 ns (range observed for several different Pgp preparations). As Pgp contains 11 Trp residue distributed over different domains, the decay complexity is not surprising, and in fact, one may even expect a distribution of fluorescence lifetimes. Therefore, the Pgp fluorescence decay depicted in FIG. 3A was also analyzed in terms of a lifetime distribution with the Exponential Series Method (ESM) (30). This analysis resulted in a bimodal distribution with the average lifetime values of 0.73 and 4.2 ns, thus being very close to the values recovered from the discrete double exponential fit (FIG. 3B). The amplitude-weighted average lifetime for Pgp was calculated as 2.4 ns.

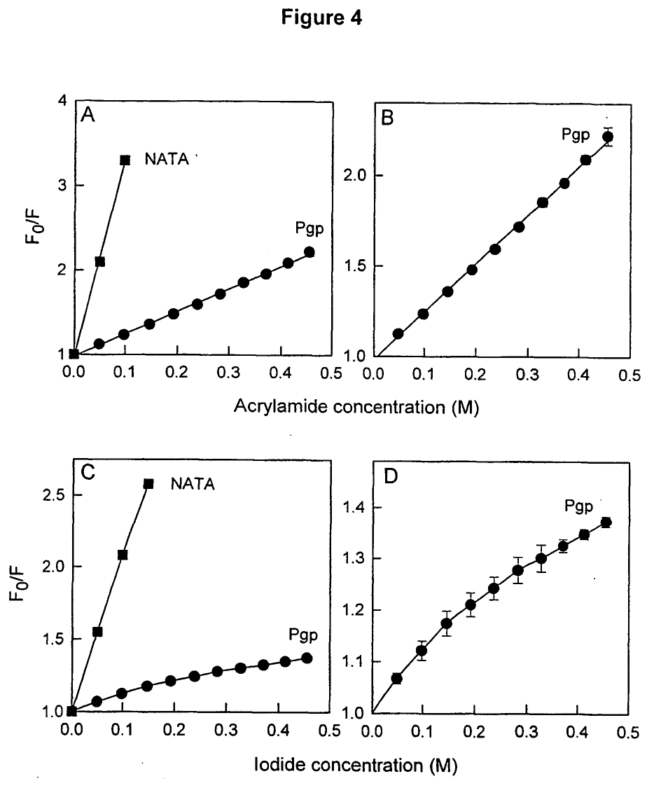

Quenching of Trp residues by Acrylamide and Iodide. Acrylamide is an excellent neutral quencher that is sensitive to exposure of Trp residues in proteins. Quenching of Trp fluorescence of Pgp was measured over the range of 0-0.5 M, and compared to that of the soluble Trp analogue, NATA (FIGS. 4A and B). ATPase assays showed that acrylamide had little effect on hydrolysis of ATP by Pgp up to a concentration of 0.3 M, with only a small decline in catalytic activity of ˜20% taking place over the range 0.3-0.5 M (data not shown). Thus, even relatively high concentrations of the quencher are expected to have only limited effects on Pgp conformation. The linear Stern-Volmer plot (an expanded plot is shown in FIG. 4B) suggests that only a single class of Trps within Pgp is quenched, and they are all equally accessible to quencher. Slight shifts in the emission maximum were noted as the concentration of acrylamide increased up to 0.3 M, possibly related to the small loss of enzymatic activity noted above. The Stern-Volmer constant (determined from the slope of the plot) for Pgp was 2.61±0.022 M⁻¹, compared to 23.1±0.35 M⁻¹ for NATA; the ˜9-fold lower K_{SV} value indicates that the Trp residues contributing to fluorescence emission in the transporter are largely buried. From K_{SV}, assuming that quenching was governed by a collisional mechanism, we calculated the biomolecular quenching constant, k_{q}, which is a measure of the intrinsic susceptibility of the Trp residues of Pgp to collisional quenching by acrylamide. The value of 1.1×10⁹ M⁻¹s⁻¹, compared to that of 6.2×10⁹ M⁻¹s⁻¹ for NATA, again indicates that the Trp residues in Pgp are relatively inaccessible.

Preliminary double-exponential decay analysis showed no trend for individual Pgp Trp residues as a function of acrylamide concentration. The set of decay curves was thus analyzed with the double-exponential global analysis program, where the lifetimes were constrained to be the same for all decay files, and only pre-exponential factors were free-floating (Table 1). The ratio of pre-exponential factors (a₁/a₂) recovered from the global analysis increased dramatically with the addition of acrylamide. Overall, this behavior implies that acrylamide molecules are trapped in pockets in close proximity to the Trp residues, and quenching is instantaneous. In contrast, NATA showed a large decrease in the fluorescence lifetime with increasing acrylamide concentrations (Table 1). The Stern-Volmer quenching constant calculated from the lifetime data was 17.0 M⁻¹, indicating that dynamic quenching is a major mechanism accounting for the observed decrease in the steady-state fluorescence for NATA. However, the pre-exponential factor is also affected, suggesting that static quenching makes an additional contribution (Table 1).

Additional information on Trp location can be provided by quenching with ionic species. Heavy atoms such as I cannot penetrate the nonpolar interior of proteins, and selectively quench only surface Trp residues. I was a very poor quencher of Trp residues within Pgp compared to NATA (FIGS. 4C and D). The quenching plot (shown in expanded form in FIG. 4D) was non-linear with a downward curve, suggesting that two classes of Trp residues exist, one of which is highly inaccessible to I. The K_{SV} value for I quenching of NATA was 10.9±0.053 M⁻¹, ˜8.5-13-fold higher than the slope of the plot for Pgp over the concentration range 0-0.5 M (0.84±0.020 to 1.27±0.033 M⁻¹). These results indicate that there are two classes of Trp residues, both largely buried, either within the membrane or the protein structure.

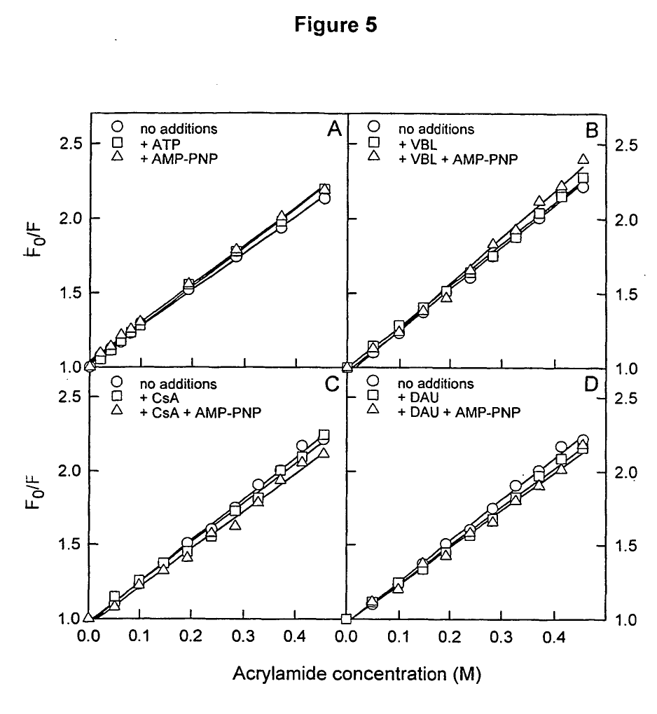

Acrylamide Quenching in the Presence of Nucleotides and Drug Substrates. Acrylamide quenching of Trp residues was carried out in the presence of saturating concentrations of nucleotides (both hydrolysable and non-hydrolysable), various drug substrates, and combinations of the two. Significant conformational changes arising from substrate binding would be expected to result in altered Trp accessibility, and changes in the values of K_{SV} for quenching. As shown in FIG. 5A, addition of saturating concentrations of either ATP or the non-hydrolysable analogue AMP-PNP, resulted in only very small changes in the slope of the Stern-Volmer plot, indicating a slight increase in accessibility of Trp residues to solvent. Addition of vinblastine gave a similarly small increase in K_{SV}, which was increased further by the addition of AMP-PNP (FIG. 5B). Cyclosporin A (FIG. 5C) and daunorubicin (FIG. 5D) also showed small changes in the slope of the plots, but in the opposite direction, with addition of drug resulting in reduced accessibility of Trp residues to solvent, and addition of AMP-PNP causing a further reduction. These results suggest that only small conformational changes affecting the solvent accessibility of Trp residues of Pgp take place on binding of drugs and nucleotides.

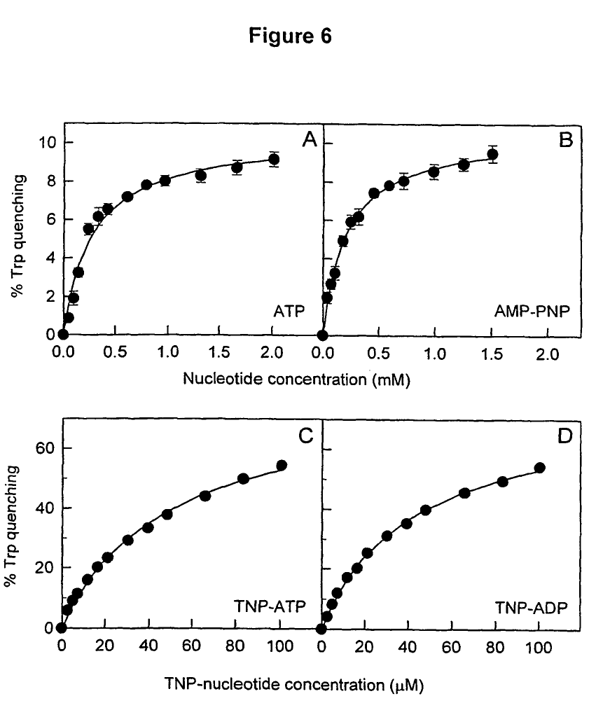

Quenching of Trp residues by Nucleotide Binding. Binding of substrates to proteins can often cause quenching of intrinsic Trp fluorescence. Addition of ATP (FIG. 6A) and ADP (data not shown) to Pgp resulted in saturable, concentration-dependent quenching of Trp fluorescence. Fitting of the quenching data to an equation representing interaction with a single type of binding site resulted in the estimation of two parameters, the dissociation constant for binding, K_{d}, and the maximal quenching reached at saturation, ΔFₘₐₓ. Values of K_{d} for ATP and ADP were around 0.3 mM (Table 2), which is almost identical to the values of K_{d} estimated from quenching of MIANS-labeled Pgp (8), and very similar to the K_{M} for ATP hydrolysis (27). Thus, Trp residues within Pgp are sensitive to nucleotide binding, and quenching data can give quantitative estimates of binding affinity. Binding of the non-hydrolysable analog AMP-PNP gave similar results (FIG. 6B), with a K_{d} of 0.21 mM. The change in Trp fluorescence appears to be brought about by nucleotide binding, rather than hydrolysis, since both hydrolysable (ATP) and non-hydrolysable molecules (ADP and AMP-PNP) gave similar values for ΔFₘₐₓ of ˜10%.

Binding of fluorescent TNP-nucleotides resulted in very strong quenching of Trp fluorescence (FIG. 6C and D), with ΔFₘₐₓ values approaching ˜80% (Table 2). The estimated K_{d} values of 51 μM and 44 μM for TNP-ATP and TNP-ADP, respectively, agree well with values obtained previously by enhancement of TNP fluorescence following binding of the fluorescent nucleotides to Pgp (36).

Quenching of Trp Residues by Binding of Drug and Peptide Substrates. Addition of various drug and peptide substrates also resulted in saturable, concentration dependent quenching of Trp fluorescence (FIG. 7). Two different quenching patterns were observed. Some substrates, such as the cyclic peptide modulator cyclosporin A (FIG. 7B), gave essentially monophasic quench curves that could be fitted to a single K_{d} value. Other drugs, such as the phosphoinositide-3-kinase inhibitor, LY294002 (37) (FIG. 7C), and the modulator trifluoperazine (FIG. 7D) gave rise to clearly biphasic curves, which were fitted to two different K_{d} values. Biphasic curves have been observed for quenching of MIANS-labeled Pgp by drugs, and were interpreted as indicating the existence of two different drug binding sites of differing affinity (32). The quench curve for some drugs, such as vinblastine (FIG. 7A) showed some biphasic character, and could be fitted to either a single value of K_{d}, or two values with affinities that were close to each other in magnitude. All of the linear and cyclic peptides tested gave monophasic quench curves, implying that they may only bind to a single site within Pgp, whereas many of the drug substrates showed biphasic characteristics, suggesting multiple binding sites (Table 2).

The estimated values of K_{d} and ΔFₘₐₓ for many Pgp substrates in different structural classes are shown in Table 2. In general, the binding affinities obtained by quenching of Trp fluorescence are very similar to those estimated from quenching of the fluorescence of MIANS-Pgp (6, 7). However, the use of Trp quenching has some additional advantages. Certain substrates are highly fluorescent in the same wavelength range as MIANS, making the estimation of K_{d} values using that method impossible. The phosphoinositide-3-kinase inhibitor LY294002 falls into the latter category. It has been confirmed that this compound is in fact a Pgp substrate by independent functional experiments; it blocks colchicine transport by Pgp in vesicle systems and stimulates Pgp ATPase activity 4-fold at a concentration of 5-10 μM (P. Lu and F. J. Sharom, unpublished data).

The values of ΔFₘₐₓ covered a wide range for different substrates, from 4-100%. Some compounds, notably the rhodamine dyes, produced very strong quenching, leading to values of ΔFₘₐₓ approaching 100%, e.g. rhodamine 123 and rhodamine B (Table 2). In general, non-peptide drugs showed ΔFₘₐₓ values of at least 15%. The hydrophobic peptide substrates and modulators, both linear and cyclic, all displayed relatively low maximal quenching, with ΔFₘₐₓ values in the range 4-6%. The monophasic nature of the quenching, and the low quenching efficiency by comparison with drug substrates, suggest that peptides may interact with Pgp in a different region from the other non-peptide drugs.

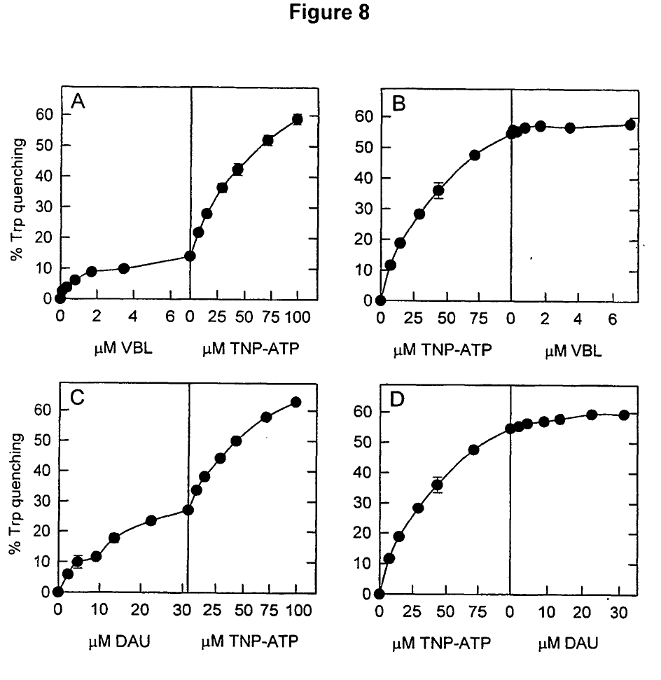

Previous work has shown that ATP and drugs bind to MIANS-Pgp in a random order (8). However, MIANS-labeled protein is catalytically inactive, and is blocked from proceeding through the catalytic cycle following substrate binding. The quenching of Trp residues in native catalytically-active Pgp was therefore investigated, following sequential titration with nucleotide and two different drugs, in either order. FIG. 8A shows the results obtained by titration with vinblastine to saturation, followed by titration of TNP-ATP, whereas FIG. 8B displays the data for the titration carried out in the reverse order. TNP-ATP can be hydrolysed by Pgp, but at a lower rate than unmodified ATP (36). Following saturation with vinblastine, titration with TNP-ATP produces the same characteristically large saturable quenching observed for the nucleotide alone, with a K_{d} of 64 μM, similar to the value of 51 μM observed in the absence of drug. Thus, the affinity of nucleotide binding is changed only slightly by prior binding of vinblastine. When TNP-ATP is bound to Pgp first to give a large amount of quenching, subsequent titration with vinblastine gives a much smaller increase in quenching than is observed with drug alone. Similar results were seen for dual titrations of TNP-ATP and daunorubicin (FIGS. 8C and D). Titration with TNP-ATP following daunorubicin binding resulted in a large degree of quenching, with a K_{d} value of 65 μM, whereas if TNP-ATP was bound first, subsequent daunomycin binding gave a very small increase in quenching. For vinblastine and daunorubicin, the combination of drug and TNP-ATP resulted in ΔFₘₐₓ values similar to those seen for TNP-ATP alone, i.e. quenching by substrate and nucleotide was not additive. These results suggest that a fixed maximal level of quenching is reached after binding of a combination of nucleotide and drug substrate.

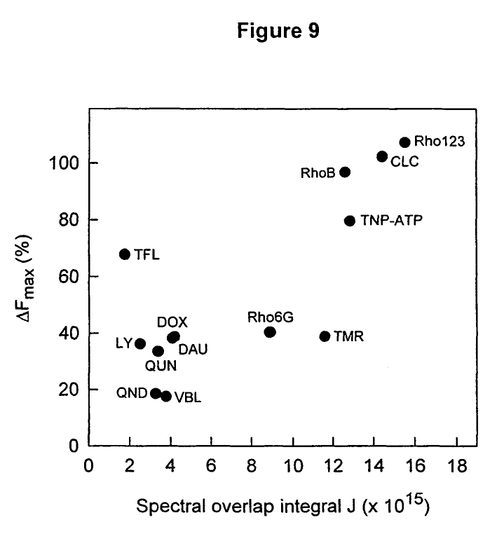

Mechanism of quenching by nucleotides, drugs and peptides. The mechanism of quenching of Pgp Trp residues by nucleotides, drugs and hydrophobic peptides is of interest in that it may provide important insights into the relative locations of the different domains of the transporter. Since Pgp fluorescence is very highly quenched by TNP-ATP/ADP, a large fraction of the Trp residues contributing to fluorescence emission may be located close to the nucleotide binding site. Quenching could take place either by a direct interaction (static quenching) or by FRET, since there is substantial overlap between the emission spectrum of Trp and the absorption spectrum of TNP-ATP. FRET is expected to result in lower fluorescence lifetimes for the Trp residues (31). However, lifetime measurements showed essentially no change in Trp lifetimes following saturation with TNP-ATP (Table 3). These results indicate that quenching takes place largely by a static mechanism resulting from direct binding. Fluorescence lifetime measurements also showed no significant decrease in Trp lifetime following saturation with ATP, AMP-PNP, and various drugs; in fact, the lifetime increased slightly in the presence of some compounds (Table 3). These results indicate that quenching by these compounds also takes place by a static, direct binding mechanism, rather than energy transfer. To further explore the possible relationship between quenching and energy transfer, the spectral overlap integral, J, for Trp was calculated and the various nucleotides and drugs. As shown in FIG. 9, there is no obvious relationship between the value of ΔFₘₐₓ (as an indicator of quenching efficiency) and J. Some compounds with quite low overlap integrals, such as trifluoperazine, cause relatively efficient quenching of Trp residues. It appears that static quenching as a result of direct binding is the major contributor to Trp quenching, which suggests that both nucleotides and drug substrates bind to a location within the protein that is close to the emitting Trp residues. It is interesting that compounds such as rhodamine 123, rhodamine B and TNP-ATP, which contain aromatic rings, were the most efficient quenchers of Trp fluorescence. If quenching results from direct binding of substrates close to Trp residues, it might be expected that interactions such as π-π stacking would be more likely for aromatic substrates.

Discussion

Based on a topology model (FIG. 1) similar to that proposed for the murine mdr3 protein (34), the 8 Trp residues in the N-half of hamster class I Pgp are located in the N-terminal tail (Trp44), in TM2, TM4 and TM5 (Trp133, Trp229 and Trp312 respectively), in the first cytoplasmic loop (Trp159), in the second extracellular loop (Trp209), and in the linker region immediately following NB1 (Trp695 and Trp705). The C-half of Pgp contains 3 Trp residues; Trp800 in cytoplasmic loop 3, Trp852 in extracellular loop5, and Trp1105 between the Walker A and B motifs of NB2. The intrinsic fluorescence spectrum of Pgp attributable to Trp residues indicates that all are located in a relatively nonpolar local environment. Acrylamide quenching experiments indicated a single class of Trps, all located in a very similar environment characterized by low accessibility to aqueous solvent. Thus it appears that the Trp residues contributing to the fluorescence emission spectrum of Pgp may be located within hydrophobic or membrane-bound regions of the protein. The value of the Stern-Volmer constant, K_{SV}, for quenching by acrylamide (2.6 M⁻¹) also suggests that the emitting Trp residues have low solvent accessibility. By comparison, the values of K_{SV} for acrylamide quenching of intrinsic Trp fluorescence of the plasma membrane and SERCA1 Ca²⁺-ATPases were 2.9 and 1.9 M⁻¹, respectively. The Trp residues in both of these proteins are mostly contained within the membrane domains. Quenching with I also indicated that the Trps of Pgp are poorly accessible to quencher, and suggested the existence of two classes of residues, one of which was highly inaccessible to this reagent, and thus largely buried. The I quenching results reported herein differ from those of Sonveaux et al. (21), who reported a linear Stern-Volmer plot, with a high degree of quenching in the presence of ATP, more typical of a denatured protein (for more discussion of this point, see (6)).

The only Trp residue for which independent fluorescence information is available to date is Trp1106 of the mouse mdr3 protein (equivalent to hamster Trp1105) located within NB2, which was expressed as a separate domain (25). It exhibited an emission maximum at 328 nm, indicating a hydrophobic environment. Thus the three Trps in TM helices, plus Trp 1105, would be expected to display spectra characteristic of a nonpolar environment. Nothing is known about the polarity of the local environment surrounding the Trps in short extracellular loops (Trp209 and 852) or in cytoplasmic regions (Trp44, 159, 695, 705, and 800), where folding into small domains may be possible. However, it seems reasonable to assume that at least some of these residues would be located in a polar environment accessible to solvent. The fact that this is not observed experimentally suggests that these residues may be quenched. Two common quenchers of Trp fluorescence are water and peptide bonds, as well as several amino acid side chains. Chen and Barkley recently identified the amino acid side chains that can quench Trp fluorescence in proteins (38); they include Gin, Asn, Glu, Asp, Cys, and His (which quench by excited-state electron transfer) as well as Lys and Tyr (which quench by excited-state proton transfer). Thus several of the Trp residues in Pgp might well be quenched by one or more of these mechanisms. The fact that the quantum yield of the Trp residues in Pgp was low (0.03) also suggests the existence of internal quenching.

The lifetime of Trp residues in proteins varies from a few hundred ps to 9 ns. Lifetime measurements of purified Pgp indicated the presence of two components, one with a shorter lifetime (0.6-1 ns), and the other major component with a longer lifetime of ˜4 ns. It is tempting to speculate that these might represent two classes to Trps, those buried deeply in TM domains and those in more accessible regions. However, in a number of studies, even single Trp proteins have been observed to show multiple lifetime components, making the time-resolved data difficult to interpret in terms of individual residues. The Trp monomer in solution has been a challenging object of numerous photophysical studies due to its non-exponential behavior (39-41). The complexity of Trp fluorescence decay has been attributed mainly to the existence of rotamers with different orientations of the indole ring relative to the carboxylic and amino groups. In proteins, different conformers may in addition be subjected to various degrees of quenching by intramolecular groups within the protein matrix, such as disulfide bonds and carbonyl groups. Protein matrix reorientation around the relatively polar excited indole chromophore may also contribute to the observed non-exponential behavior. The dynamic origin of four lifetime components for the single Trp horse heart apocytochrome c has been well documented (42). Further experiments are needed to shed more light on the origin of the two lifetime components of Trp-rich Pgp. In this regard, the ability to examine the behavior of single Trp Pgp mutants would be of immense advantage. The stage has been set for production of these types of engineered proteins with the recent report of the construction of a functional Trp-less Pgp (43).

To investigate possible conformational changes taking place on binding of substrates, acrylamide quenching studies were carried out in the presence of nucleotides, drugs, and combinations of the two. Only very small changes in K_{SV} were noted following saturation with any of these molecules, arguing against major conformational changes that alter the environment of Trp residues following substrate binding. These results support previous observations that a MIANS label within the catalytic sites of Pgp is only slightly less accessible to solvent (K_{SV} was reduced by ˜10%) when the nucleotide binding sites are occupied (36), suggesting that the conformational change induced by nucleotide binding is small. In contrast, it was recently reported that K_{SV} values for acrylamide quenching of purified Pgp were substantially altered following nucleotide and drug binding (22). However, this study looked at only a very narrow range of acrylamide concentrations (0-0.08 M), and the fluorescence changes observed were only a maximum of 4-10% at the highest concentration. Moreover, under some conditions, no acrylamide quenching at all was noted (K_{SV}=0, a very unusual observation), suggesting that caution should be observed when interpreting these data.

Pgp Trp residues were quenched in a saturable fashion by binding of various nucleotides, both hydrolysable (ATP) and non-hydrolysable (ADP, AMP-PNP). Since all gave similar changes in fluorescence (ΔFₘₐₓ of ˜10%), the change in Trp fluorescence appears to be brought about by nucleotide binding, rather than hydrolysis. The K_{d} values estimated from fitting the data to an equation describing binding to a single type of site were in the range 0.2-0.3 mM, in good agreement with values obtained from MIANS-Pgp quenching (7, 8), and close to the K_{M} for ATP hydrolysis (27). The fluorescent nucleotide analogs ATP-ATP and TNP-ADP caused highly efficient quenching of the Pgp fluorescence, with ΔFₘₐₓ values approaching 80%. Again, saturable titration of these nucleotide analogs gave K_{d} values in good agreement with determinations made by other methods (36). Binding of several fluorescent-nucleotides (including TNP-ATP) to helicase DnaB also results in strong quenching of the Trp fluorescence (13). It was shown that in this case quenching arises from highly efficient energy transfer to the fluorescent derivative from Trp residues, which are clustered together near the nucleotide binding sites. However, in the case of Pgp, the absence of any significant decrease in the Trp fluorescence lifetime indicates that quenching by TNP-nucleotide takes place by a static, direct binding mechanism, rather than FRET.

Pgp Trp residues were also highly sensitive to binding of substrates, with various drugs, modulators and hydrophobic peptides causing saturable concentration-dependent quenching of the fluorescence. Trp quenching by some substrates displayed biphasic characteristics, suggesting. the existence of two drug binding sites with different affinities. Similar results for binding of three drugs to MIANS-labeled Pgp reconstituted into lipid bilayers have been reported (32). Sequential titration with both drug and nucleotide confirmed previous observations (made with catalytically inactive MIANS-Pgp) that binding of these substrates can take place in any order (8). The observation that quenching caused by TNP-ATP and vinblastine was not additive suggests that a maximal level of quenching is reached in the loaded transporter, perhaps reflecting the attainment of a transport competent conformation.

The presence of phospholipid proved beneficial for observation of nucleotide and drug-induced quenching of Trp fluorescence with some drugs, suggesting that it may play a modulatory role in substrate binding. However, the addition of lipid did not produce a significant change in the Trp emission maximum or fluorescence lifetimes. It has been previously reported that quenching of MIANS-labeled Pgp by substrates also benefited from the presence of phospholipids (8), and more recently, the apparent affinity of drug binding depends on the lipid environment has been demonstrated (32). Lipids may help maintain Pgp conformation, as well as provide a “pool” into which drugs can partition to access the binding site(s), which appear to lie within the membrane-spanning regions of the protein (for a review, see (44)). The use of lipids was not necessary with all drugs. For example, the compound Hoechst 33342 (see Table 2) gave good titration curves with or without lipid.

In the case of Pgp, the strong quenching by some drugs (such as rhodamine dyes and trifluoperazine) far exceeds that expected from the very small measured changes in fluorescence lifetime (Table 3), many of which were increased, rather than decreased as expected if FRET were responsible for the quenching. These results indicate that the major contribution to quenching is via a static mechanism resulting from direct binding of the compounds close to the Trp residues. This implies that the Trps contributing to Pgp intrinsic fluorescence are located relatively close to the drug binding sites. Since these residues are probably largely membrane-bound, based on their fluorescence emission maximum and their quenching properties, this conclusion is consistent with the idea that drug binding takes place within the membrane-spanning regions of Pgp.

Table 2 shows a wide range of values for ΔFₘₐₓ (which reflects the quenching efficiency), ranging from 4-6% for hydrophobic peptides to essentially 100% for some of the rhodamine dyes. If quenching results from direct binding of drug close to Trp residues located within the membrane, then the magnitude of ΔFₘₐₓ may reflect both the intrinsic quenching properties of the drug itself (i.e. its chemical makeup) as well as the proximity of its binding site to the Trp residues. In addition, the presence of multiple drug binding sites, each of which would likely have different distance relationships with the Trp residues, may contribute to the wide range of quenching efficiencies observed experimentally.

It is possible that drugs could quench Pgp Trp residues by an indirect mechanism not involving close contact. For example a relatively large conformational change taking place in the membrane-spanning regions of Pgp induced by drug binding might move chemical groups within the protein to positions where they could efficiently quench Trp residues. Such a change seems unlikely, given the extremely small changes in Trp accessibility to acrylamide quenching that were noted following binding of three different substrates (FIG. 4).

Quenching of Trp fluorescence is also observed for nucleotides, raising the question of the mechanism by which this takes place. In the case of unmodified molecules such as ATP, ADP, and AMP-PNP, the maximal quenching, although relatively low (in the 10% range), is larger than that of linear and cyclic peptide substrates, and only slightly smaller than that of vinblastine and quinidine. Acrylamide quenching studies using MIANS-Pgp have already suggested that only small changes in the conformation of the NB domains takes place on nucleotide binding (36). Binding of either ATP or AMP-PMP to Pgp resulted in insignificant changes in Trp accessibility to acrylamide (FIG. 4), suggesting that conformational changes in the membrane-bound domains of Pgp on nucleotide binding that affect the Trp residues are also very small. Since a large conformational change is improbable, it seems likely that the Trp quenching observed for ATP and ADP also arises from a direct static quenching effect, which would imply that the site for nucleotide binding is close to the membrane-bound Trp residues, and by extension, close to the drug binding sites. This proposal is supported by the very strong Trp quenching (ΔFₘₐₓ) observed for TNP nucleotides. Lifetime measurements, and the absence of sensitized emission from TNP-ATP, indicated that TNP-ATP quenching did not take place as a result of energy transfer. Since ATP and TNP-ATP bind competitively to the same site on Pgp (36), the chemical nature of the TNP group presumably accounts for the high level of quenching observed with the modified nucleotide. The separately-expressed C-terminal NB domain of Pgp contains a single Trp residue, which has been shown to “report” nucleotide binding by quenching. However, ˜80% quenching of the intrinsic fluorescence of full-length Pgp containing 11 Trp residues cannot be accounted for by the proximity of a single residue to the bound nucleotide. Taken together, our results strongly suggest that both the drug and ATP binding sites of Pgp are located in close proximity to each other, and to the membrane-bound Trp residues, indicating that the regions of the molecule involved in binding and transport may be packed together quite compactly. Such proximity between the two types of binding sites would facilitate coupling between drug transport and ATP hydrolysis.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE I
Acrylamide Quenching Effect on Pgp Trp and
NATA Fluorescence Lifetimes

| sample | a₁ | t₁ (ns) | a₂ | t₂ (ns) | a₁/a₂ | <Tₐᵥ> (ns) |
|---|---|---|---|---|---|---|
| Pgp alone^{a} | 0.276 | 0.64 | 0.314 | 4.0 | 0.879 | 2.43 |
| Pgp + acrylamide | | | | | | |
| 0.098 M | 0.302 | ″ | 0.210 | ″ | 1.43 | 2.02 |
| 0.192 M | 0.215 | ″ | 0.136 | ″ | 1.58 | 1.94 |
| 0.283 M | 0.270 | ″ | 0.149 | ″ | 1.81 | 1.83 |
| 0.370 M | 0.194 | ″ | 0.0921 | ″ | 2.11 | 1.72 |
| 0.455 M | 0.184 | ″ | 0.0938 | ″ | 1.96 | 1.78 |

| | | | |
|---|---|---|---|
| sample^{b} | a₁ | t₁ (ns) | t/t₀ |
| NATA alone | 0.949 | 2.63 | 1.00 |
| NATA + acrylamide | | | |
| 0.098 M | 0.680 | 1.03 | 0.392 |
| 0.192 M | 0.456 | 0.616 | 0.234 |
| 0.283 M | 0.302 | 0.497 | 0.189 |
| 0.370 M | 0.245 | 0.351 | 0.133 |

^{a}Two-exponential global analysis was carried out, recovering the same two lifetimes of 0.64 and 4.0 ns for all decays. aᵢ is the pre-exponential value; tᵢ is the fluorescence lifetime; <tₐᵥ> is the amplitude average lifetime.
^{b}Decays were fitted to a single exponential function.

TABLE 2
Binding Parameters for Quenching of Pgp Trp Fluorescence
by Nucleotides, Drug and Peptide Substrates

| substrate | K_{d1} (μM)^{a} | K_{d2} (μM)^{a} | ΔFₘₐₓ₁ (%)^{a} | ΔFₘₐₓ₂ (%)^{a} |
|---|---|---|---|---|
| nucleotides | | | | |
| ATP | 280 | | 10.4 | |
| ADP | 330 | | 9.46 | |
| AMP-PNP | 210 | | 10.6 | |
| TNP-ATP | 50.6 | | 79.6 | |
| non-peptide drugs | | | | |
| colchicine | 74.9 ± 8.6 | | 102.2 ± 5.7 | |
| mitelfosine | 20.6 ± 3.3 | | 12.0 ± 1.0 | |
| D21266 | 20.1 ± 3.4 | | 13.0 ± 1.15 | |
| (Zentaris AG, Frankfurt) | | | | |
| D-20133 | 12.8 ± 2.1 | | 12.3 ± 0.89 | |
| (Zentaris AG, Frankfurt) | | | | |
| quinine | 12.5 ± 2.6 | | 33.5 ± 2.2 | |
| quinidine | 7.76 ± 0.73 | | 18.5 ± 0.46 | |
| rhodamine 123 | 6.81 ± 0.71 | 29.8 ± 1.0 | 47.5 ± 2.5 | 107.2 ± 1.8 |
| LY294002 | 6.29 ± 0.88 | 11.51 ± 1.5 | 29.7 ± 1.6 | 36.2 ± 2.0 |
| doxorubicin | 5.44 ± 0.55 | 21.6 ± 3.2 | 19.5 ± 0.90 | 38.7 ± 3.0 |
| rhodamine B | 4.00 ± 0.45 | 33.16 ± 3.1 | 28.8 ± 1.5 | 96.8 ± 4.8 |
| daunorubicin | 2.97 ± 0.38 | 10.7 ± 1.7 | 22.7 ± 1.3 | 38.2 ± 2.4 |
| propafenone GP12 | 2.58 ± 0.41 | | 8.97 ± 0.36 | |
| trifluoperazine | 1.56 ± 0.17 | 16.8 ± 1.6 | 18.9 ± 0.87 | 67.7 ± 4.1 |
| Hoechst 33342 | 2.61 ± 0.182 | | 57.2 ± 1.97 | |
| tetramethylrosamine | 0.81 ± 0.25 | 2.70 ± 0.20 | 21.0 ± 3.3 | 32.8 ± 0.88 |
| propafenone GP02 | 0.75 ± 0.096 | | 9.71 ± 0.27 | |
| rhodamine 6G | 0.57 ± 0.053 | 4.27 ± 0.54 | 24.0 ± 0.60 | 40.4 ± 1.9 |
| vinblastine | 0.50 ± 0.08 | | 17.5 ± 0.82 | |
| propafenone GP03 | 0.37 ± 0.030 | | 8.59 ± 0.15 | |
| propafenone GP366 | 0.25 ± 0.037 | | 7.76 ± 0.29 | |
| propafenone GP576 | 0.062 ± 0.004 | | 9.27 ± 0.11 | |
| peptides and peptide-based drugs | | | | |
| pepstatin A | 9.53 ± 0.78 | | 4.46 ± 0.073 | |
| valinomycin | 0.72 ± 0.084 | | 4.14 ± 0.086 | |
| reversin 205 | 0.37 ± 0.037 | | 5.41 ± 0.13 | |
| cyclosporin A | 0.30 ± 0.021 | | 4.44 ± 0.088 | |
| reversin 201 | 0.23 ± 0.02 | | 6.18 ± 0.10 | |
| PSC833 | 0.081 ± 0.0071 | | 4.02 ± 0.079 | |
| ^{a}Dissociation constants for binding were obtained by fitting of the Trp quenching data to an equation for either a single binding site (K_{d1}), or (where appropriate) two binding sites, one of high affinity (K_{d1}) and one of lower affinity (K_{d2}). ΔFₘₐₓ values were also obtained from fitting of the quenching data. Where a biphasic quenching curve was observed, the values for both the higher (Fₘₐₓ₁) and the lower affinity (ΔFₘₐₓ₁) and | | | | |
| #the lower affinity (ΔFₘₐₓ₂) binding component are shown. | | | | |

TABLE 3
Trp Fluorescence Lifetimes in the Absence
and Presence of Nucleotides and Drugs

| | a₁ | t₁ (ns) | a₂ | t₂ (ns) | <tₐᵥ> (ns) |
|---|---|---|---|---|---|
| Pgp alone | 0.55 | 0.96 | 0.74 | 4.1 | 2.76 |
| nucleotide binding | | | | | |
| 50 μM TNP-ATP | 0.69 | 1.0 | 0.42 | 4.0 | 2.14 |
| drug binding | | | | | |
| 10 μM vinblastine | 1.07 | 1.1 | 0.51 | 4.4 | 2.17 |
| 40 μM daunorubicin | 0.71 | 1.3 | 0.52 | 4.4 | 2.61 |
| 30 μM trifluoperazine | 0.72 | 1.1 | 0.35 | 4.3 | 2.15 |
| 10 μM tetramethylrosamine | 2.43 | 0.55 | 1.1 | 0.50 | 3.9 |
| 50 μM rhodamine 123 | 0.65 | 1.5 | 0.43 | 4.6 | 2.73 |
| 150 mM colchicine | 0.66 | 1.4 | 0.39 | 4.7 | 2.63 |

^{a}Two-exponential global analysis was carried out, recovering the same two lifetimes of 0.96 and 4.1 ns for all decays. aᵢ is the pre-exponential value; tᵢ is the fluorescence lifetime; <tₐᵥ> is the amplitude average lifetime.

### FULL CITATIONS FOR REFERENCES REFERRED TO IN THE SPECIFICATION

1. Holland, I. B., and Blight, M. A. (1999) J. Mol. Biol. 293, 381-399.

2. Doige, C. A., and Ames, G. F. (1993) Annu. Rev. Microbiol. 47, 291-319.

3. Schinkel, A. H. (1999) Adv. Drug Deliv. Rev. 36, 179-194.

4. Chan, H. S., Grogan, T. M., DeBoer, G., Haddad, G., Gallie, B. L., and Ling, V. (1996) Eur. J. Cancer 32A, 1051-1061.

5. Bradshaw, D. M., and Arceci, R. J. (1998) J. Clin. Oncol. 16, 3674-3690.

6. Sharom, F. J., Liu, R. H., and Romsicki, Y. (1998) Biochem. Cell Biol. 76, 695-708.

7. Sharom, F. J., Liu, R. H., Romsicki, Y., and Lu, P. H. (1999) Biochim. Biophys. Acta 1461, 327-345.

8. Liu, R., and Sharom, F. J. (1996) Biochemistry 35, 11865-11873.

9. Sharom, F. J., Lu, P., Liu, R., and Yu, X. (1998) Biochem. J. 333, 621-630.

10. Weber, J., Wilke-Mounts, S., Hammond, S. T., and Senior, A. E. (1998) Biochemistry 37, 12042-12050.

11. Lopez, M. M., and Kosk-Kosicka, D. (1998) Biophys. J. 74, 974-980.

12. Gryczynski, I., Wiczk, W., Inesi, G., Squier, T., and Lakowicz, J. R. (1989) Biochemistry 28, 3490-3498.

13. Bujalowski, W., and Klonowska, M. M. (1994) J. Biol. Chem. 269, 31359-31371.

14. Guixe, V., Rodriguez, P. H., and Babul, J. (1998) Biochemistry 37, 13269-13275.

15. Mus-Veteau, I., Pourcher, T., and Leblanc, G. (1995) Biochemistry 34, 6775-6783.

16. Weitzman, C., Consler, T. G., and Kaback, H. R. (1995) Protein Sci. 4, 2310-2318.

17. Pawagi, A. B., Wang, J., Silverman, M., Reithmeier, R. A., and Deber, C. M. (1994) J. Mol. Biol. 235, 554-564.

18. Zheleznova, E. E., Markham, P., Edgar, R., Bibi, E., Neyfakh, A. A., and Brennan, R. G. (2000) Trends Biochem. Sci. 25, 39-43.

19. Zheleznova, E. E., Markham, P. N., Neyfakh, A. A., and Brennan, R. G. (1999) Cell 96, 353-362.

20. Popkov, M., Lussier, I., Medvedkine, V., Estève, P. O., Alakhov, V., and Mandeville, R. (1998) Eur. J. Biochem. 251, 155-163.

21. Sonveaux, N., Shapiro, A. B., Goormaghtigh, E., Ling, V., and Ruysschaert, J. M. (1996) J. Biol. Chem. 271, 24617-24624.

22. Sonveaux, N., Vigano, C., Shapiro, A. B., Ling, V., and Ruysschaert, J. M. (1999) J. Biol. Chem. 274, 17649-17654.

23. Baubichon-Cortay, H., Baggetto, L. G., Dayan, G., and Di Pietro, A. (1994) J. Biol. Chem. 269, 22983-22989.

24. Dayan, G., Baubichon-Cortay, H., Jault, J. M., Cortay, J. C., Deleage, G., and Di Pietro, A. (1996) J. Biol. Chem. 271, 11652-11658.

25. Conseil, G., Baubichon-Cortay, H., Dayan, G., Jault, J. M., Barron, D., and Di Pietro, A. (1998) Proc. Natl. Acad. Sci. U.S.A 95, 9831-9836.

26. Sharom, F. J., Yu, X., Lu, P., Liu, R., Chu, J. W., Szabo, K., Muller, M., Hose, C. D., Monks, A., Varadi, A., Seprodi, J., and Sarkadi, B. (1999) Biochem. Pharmacol. 58, 571-586.

27. Sharom, F. J., Yu, X., Chu, J. W. K., and Doige, C. A. (1995) Biochem. J. 308, 381-390.

28. Groenzin, H., and Mullins, O. C. (1999) J. Phys. Chem. A 103, 11237-11245.

29. James, D. R., Siemiarczuk, A., and Ware, W. R. (1992) Rev. Sci. Instr. 63, 1710-1716.

30. Siemiarczuk, A., Wagner, B. D., and Ware, W. R. (1990) J. Phys. Chem. 94, 1661-1666.

31. Lakowicz, J. R. (1999) in Principles of fluorescence spectroscopy, Kluwer Academic Publishers, New York,

32. Romsicki, Y., and Sharom, F. J. (1999) Biochemistry 38, 6887-6896.

33. Devine, S. E., and Melera, P. W. (1994) Cancer Chemother. Pharmacol. 33, 465-471.

34. Kast, C., Canfield, V., Levenson, R., and Gros, P. (1996) J. Biol. Chem. 271, 9240-9248.

35. Romsicki, Y., and Sharom, F. J. (1998) Eur. J. Biochem. 256, 170-178.

36. Liu, R., and Sharom, F. J. (1997) Biochemistry 36, 2836-2843.

37. Vlahos, C. J., Matter, W. F., Hui, K. Y., and Brown, R. F. (1994) J. Biol. Chem. 269, 5241-5248.

38. Chen, Y., and Barkley, M. D. (1998) Biochemistry 37, 9976-9982.

39. James, D. R., and Ware, W. R. (1985) Chem. Phys. Lett. 120, 450-454.

40. Szabo, A. G., and Rayner, D. M. (1980) J. Amer. Chem. Soc. 102, 554-563.

41. Chang, M. C., Petrich, J. W., McDonald, D. B., and Fleming, G. R. (1983) J. Amer. Chem. Soc. 105, 3819-3824.

42. Vincent, M., Brochon, J. C., Merola, F., Jordi, W., and Gallay, J. (1988) Biochemistry 27, 8752-8761.

43. Kwan, T., Loughrey, H., Brault, M., Gruenheid, S., Urbatsch, I. L., Senior, A. E., and Gros, P. (2000) Mol. Pharmacol. 58, 37-47.

44. Loo, T. W., and Clarke, D. M. (1999) Biochim. Biophys. Acta 1461, 315-325.

We claim:

1. A method of assaying for compounds that interact with P-glycoprotein comprising measuring the intrinsic tryptophan fluorescence of P-glycoprotein in the presence of varying concentrations of the compound, and determining a constant describing the interaction of the compound with P-glycoprotein.

2. A method of determining the dissociation constant (K_{d}) of a compound for P-glycoprotein comprising measuring the intrinsic tryptophan fluorescence of P-glycoprotein in the presence of varying concentrations of the compound, and determining the dissociation constant.

3. The method according to any of claims 1-2, wherein the source of P-glycoprotein is mammalian.

4. The method according to claim 3, wherein the source of P-glycoprotein is selected from the group consisting of human, mouse, hamster, rat, monkey and rabbit.

5. The method according to claim 3, wherein the P-glycoprotein is obtained from a multidrug-resistant cell line.

6. The method according to claim 3, wherein the P-glycoprotein is obtained from a heterologous expression system.

7. The method according to claim 5, wherein the source of P-glycoprotein is the multidrug-resistant Chinese hamster ovary cell line CH^{R}B30.

8. The method according to any of claims 3-7, wherein the intrinsic tryptophan fluorescence is measured in the presence of varying concentrations of the compound and one or more lipids.

9. The method according to claim 8, wherein the one or more lipids is a phospholipid.

10. The method according to claim 9, wherein the phospholipid is selected from the group consisting of egg phosphatidylcholine (egg PC), dimyristoylphosphatidylcholine (DMPC), dipalmitoylphosphatidylcholine (DPPC), and palmitoylmyristoylphosphatidylcholine (PMPC).

11. The method according to claim 10, wherein the phospholipid is PMPC.

12. The method according to any of claims 8-11, wherein the intrinsic tryptophan fluorescence of P-glycoprotein is measured in the presence of at least 5 concentrations of the compound.

13. The method according to claim 12, wherein the intrinsic tryptophan fluorescence of P-glycoprotein is measured in the presence of at least 10 concentrations of the compound.

14. The method according to claim 13, wherein the intrinsic tryptophan fluorescence of P-glycoprotein is measured in the presence of at least 15 concentrations of the compound.

15. A method of assaying for compounds that interact with P-glycoprotein comprising:
(a) providing a sample of P-glycoprotein;
(b) adding a first concentration of a compound to the sample of P-glycoprotein and measuring the intrinsic tryptophan fluorescence;
(c) repeating steps (a) and (b) with a second concentration of the compound; and
(d) determining the dissociation constant (K_{d}) for the binding of the compound to P-glycoprotein, wherein the K_{d} indicates the interaction of the compound with P-glycoprotein.

16. The method according to claim 15, wherein steps (a) and (b) are repeated with at least 5 concentrations of the compound.

17. The method according to claim 15, wherein steps (a) and (b) are repeated with at least 10 concentrations of the compound.

18. The method according to claim 15, wherein steps (a) and (b) are repeated with at least 15 concentrations of the compound.

19. The method according to any of claims 15-18, wherein the intrinsic tryptophan fluorescence is measured in the presence of varying concentrations of the compound and one or more lipids.

20. The method according to claims 19, wherein the one or more lipids is a phospholipid.

21. The method according to claim 20, wherein the phospholipid is selected from the group consisting of egg phosphatidylcholine (egg PC), dimyristoylphosphatidylcholine (DMPC), dipalmitoylphosphatidylcholine (DPPC), and palmitoylmyristoylphosphatidylcholine (PMPC).

22. The method according to claim 21, wherein the phospholipid is PMPC.

23. A method of determining if a compound is a good drug candidate comprising measuring the intrinsic tryptophan fluorescence of P-glycoprotein in the presence of varying concentrations of the compound, fitting the fluorescence measurements to an equation describing binding to a single site or multiple sites and extraction of a constant describing the interaction of the compound with P-glycoprotein, wherein the value of the constant indicates whether the compound is a good drug candidate.

24. A method of determining if a compound is a good drug candidate comprising:
(a) providing a sample of P-glycoprotein in the presence one or more lipids;
(b) adding a first concentration of the compound to the sample of P-glycoprotein and measuring the intrinsic tryptophan fluorescence;
(c) repeating steps (a) and (b) with a second concentration of the compound; and
(d) determining the dissociation constant (K_{d}) for the binding of the compound to P-glycoprotein, wherein the value of K_{d} indicates whether the compound is a good drug candidate.

25. The method according to claim 24 wherein a K_{d} of greater than about 100 μM, indicates that the compound is a good drug candidate.

26. The method according to any of claims 24-25 wherein the one or more lipids is a phospholipid.

27. A method of identifying compounds that are high affinity P-glycoprotein modulators comprising measuring the intrinsic tryptophan fluorescence of P-glycoprotein in the presence of varying concentrations of the compound, fitting the fluorescence measurements to an equation describing binding to a single site or multiple sites and extraction of a constant describing the interaction of the compound with P-glycoprotein, wherein the value of the constant indicates whether the compound is a potentially effective P-glycoprotein modulator.

28. A method of identifying compounds that are high affinity P-glycoprotein modulators comprising:
(a) providing a sample of P-glycoprotein in the presence of one or more lipids;
(b) adding a first concentration of the compound to the sample of P-glycoprotein and measuring the intrinsic tryptophan fluorescence;
(c) repeating steps (a) and (b) with a second concentration of the compound; and
(f) determining the dissociation constant (K_{d}) for the binding of the compound to P-glycoprotein, wherein the value of K_{d} indicates if the compound is a potentially effective P-glycoprotein modulator.

29. The method according to claim 28, wherein, a K_{d} of less than about 10 μM indicates that the compound is a potentially effective P-glycoprotein modulator.

30. The method according to any of claims 28-29 wherein the one or more lipids is a phospholipid.

31. A method of determining if a compound may be dangerous if used in combination with a modulator of P-glycoprotein comprising measuring the intrinsic tryptophan fluorescence of P-glycoprotein in the presence of varying concentrations of the compound, fitting the fluorescence measurements to an equation describing binding to a single site or multiple sites and extraction of a constant describing the interaction of the compound with P-glycoprotein, wherein the value of the constant indicates whether the compound may be dangerous if used in combination with a modulator of P-glycoprotein.

32. A method of determining if a compound may be dangerous if used in combination with a modulator of P-glycoprotein comprising:
(a) providing a sample of P-glycoprotein in the presence of one or more lipids;
(b) adding a first concentration of the compound to the sample of P-glycoprotein and measuring the intrinsic tryptophan fluorescence;
(c) repeating steps (a) and (b) with a second concentration of the compound; and
(d) determining the dissociation constant (K_{d}) for the binding of the compound to P-glycoprotein, wherein the value of K_{d} indicates if the compound may be dangerous if used in combination with a modulator of P-glycoprotein.

33. The method according to claim 32, wherein, a K_{d} of less than about 10.0 μM indicates that the compound is potentially dangerous if used in combination with a modulator of P-glycoprotein.

34. The method according to any of claims 32-33 wherein the one or more lipids is a phospholipid.

35. A method of conducting a target discovery business comprising:
(a) providing one or more assay systems for identifying compounds by their ability to interact with P-glycoprotein, said assay systems using a method of the invention;
(b) (optionally) conducting therapeutic profiling of agents identified in step (a) for efficacy and toxicity in animals; and
(c) licensing, to a third party, the rights for further drug development and/or sales of compounds identified in step (a), or analogs thereof.

36. A method of conducting a compound screening business comprising:
(a) providing one or more assay systems for screening compounds for their ability to interact with P-glycoprotein, said assay systems using a method of the invention; and
(b) providing, to a third party, the information identified in step (a) in exchange for compensation.

37. A kit for performing the method according to any of claims 1-34 comprising an aliquot of P-glycoprotein, an aliquot of one or more lipids, and buffer.

38. The kit according to claim 37, wherein the one or more lipids is a phospholipid.

39. The kit according to claim 38, wherein the phospholipid is selected from the group consisting of egg phosphatidylcholine (egg PC), dimyristoylphosphatidylcholine (DMPC), dipalmitoylphosphatidylcholine (DPPC), and palmitoylmyristoylphosphatidylcholine (PMPC).

40. The kit according to claim 39, wherein the phospholipid is PMPC.

41. The kit according to any of claims 37-40, wherein the buffer is CHAPS buffer.

42. A compounds identified using a method according to any of claims 1-34.

43. A pharmaceutical compositions comprising a compound identified using a method according to any of claims 1-34 and a pharmaceutically acceptable carrier.

44. A method of preparing a composition comprising determining whether a compound is a modulator of P-glycoprotein using a method according to any of claims 1-34 and admixing said compound with a pharmaceutically acceptable carrier.
